(19) United States
(12) Patent Application Publication
(10) Pub. No.: US 2026/0262958 A1
(43) Pub. Date: Sep. 10, 2026

(54) PRESSURE SENSORS

(71) Applicants: Layla Khalifehzadeh, Tempe, AZ (US); Hamed Arami, Chandler, AZ (US); Seyedamin Hashemi, Tempe, AZ (US); Saman Ebrahimibasabi, Tempe, AZ (US); Mostafa Sajjadi, Tempe, AZ (US)

(72) Inventors: Layla Khalifehzadeh, Tempe, AZ (US); Hamed Arami, Chandler, AZ (US); Seyedamin Hashemi, Tempe, AZ (US); Saman Ebrahimibasabi, Tempe, AZ (US); Mostafa Sajjadi, Tempe, AZ (US)

(21) Appl. No.: 19/558,122

(22) Filed: Mar. 5, 2026

Related U.S. Application Data
(60) Provisional application No. 63/767,824, filed on Mar. 6, 2025.

(51) Int. Cl. A61B 5/03; A61B 5/00; G01L 9/00; G01L 9/12

(52) U.S. Cl. CPC A61B 5/031; A61B 5/6868; A61B 5/7271; A61B 5/742; G01L 9/0005; G01L 9/12; A61B 2562/0247; A61B 2562/028; A61B 2562/12; A61B 2562/16

(57) ABSTRACT

Disclosed herein is a pressure sensor that may include a first inductive conductor and a second inductive conductor. An example pressure sensor may include a dielectric layer positioned between the first inductive conductor and the second inductive conductor, wherein the dielectric layer includes a plurality of micro-structured elastomers comprising a mixture of organic and inorganic polymers along with an inorganic compound.

### CROSS-REFERENCE TO RELATED APPLICATIONS

This application claims priority to U.S. Provisional Patent Application No. 63/767,824 filed on Mar. 6, 2025, the entire contents of which are incorporated herein by reference.

### STATEMENT REGARDING FEDERALLY SPONSORED RESEARCH OR DEVELOPMENT

This invention was made with government support under CA234208 and EB031178 awarded by the National Institutes of Health. The government has certain rights in the invention.

### TECHNICAL FIELD

The present disclosure relates to pressure sensors, and more specifically to ultra-sensitive wireless capacitive nanocomposite-based pressure sensors.

### BACKGROUND

Wireless pressure sensing plays a role in various applications, including robotics, wearable technologies, and health monitoring. Advances in flexible electronics and materials science have made significant improvements in designing these sensors, expanding their real-world applications. Among the various pressure sensors, passive wireless capacitive pressure sensors have demonstrated potential for monitoring internal pressures, in a variety of applications such as intraocular pressure, blood pressure, bladder pressure, and intracranial pressure (ICP), holding promise for clinical applications.

Recording and interpreting biological signals are utilized to advance understanding of human physiology and enabling the timely diagnosis of disorders and abnormal changes in the body. In recent years, technological advancements have facilitated the development of in-home recording and diagnostic devices, making continuous health monitoring more accessible. This transition to home-based monitoring is particularly important in cases where early disease symptoms are not evident, yet patients require long-term observation. Monitoring in a hospital setting can be prohibitively expensive and inconvenient for both patients and healthcare providers. Among the key biological signals, pressure measurement stands out as a vital indicator in monitoring various diseases, such as monitoring of blood flow in patients suffering from high blood pressure, tracking of bladder pressure in patients with urine incontinence, intraocular pressure monitoring in glaucoma patients, and monitoring intracranial pressure (ICP) in patients experiencing traumatic brain injuries. Traditional methods of measuring internal pressure, such as catheter-based ICP monitoring, present several challenges. These methods often require bulky equipment, limit patient mobility, and necessitate specialized expertise, making them unsuitable for home care or outpatient settings.

Ultra-small wireless pressure sensors have emerged as a promising alternative, offering advantages not only in health monitoring but also in other fields such as robotics and wearable technologies. Accordingly, it is desirable to provide a pressure sensor that improves sensitivity for detecting pressure changes. Additionally, in a medical context, it is desirable to provide a pressure sensor that improves sensitivity for detecting physiological pressure changes in high-risk patients with complex conditions.

### SUMMARY

Continuous real-time postoperative pressure monitoring in organs such as the brain, heart, lungs, and bladder is critical for timely clinical diagnosis and intervention. Pressure dynamics are critical physiological indicators, with abnormal variations often signaling potential complications. Early detection of these changes enables prompt medical intervention, which is especially important in managing high-risk patients with complex conditions. This proactive approach can significantly improve patient outcomes by preventing adverse events in a timely manner.

Flexible sensors are broadly classified into active and passive types. Active sensors, though effective, typically involve complex designs and circuitry and rely on intricate power sources such as near-field communication or batteries. Moreover, data transmission in these sensors often requires Bluetooth® modules or antennas, adding to their complexity. Conversely, passive pressure sensors, which consist of passive components, are more compact, portable, and cost-effective. Pressure sensors are also categorized based on their sensing mechanisms, which include piezoelectric, resistive, and capacitive methods. While piezoelectric and resistive sensors are often prone to temperature sensitivity, capacitive pressure sensors offer superior sensitivity, flexibility, accuracy, and temperature independence. Capacitive sensors operate wirelessly through the creation of an inductor-capacitor (LC) resonator, achieved by placing a dielectric layer between two spiral-shaped traces. Changes in capacitance caused by pressure-induced alterations in the dielectric result in shifts in the resonance frequency of the resonator. This frequency shift is detected wirelessly using a pickup probe, allowing electromagnetic (EM) coupling with the sensor.

The dielectric layer in wireless capacitive pressure sensors for biomedical applications is typically made of biocompatible polymers such as polydimethylsiloxane (PDMS), styrene-butadiene-styrene (SBS), or styrene-ethylene-butylene-styrene (SEBS). These polymers are compressed in response to applied pressure, causing a distance reduction between the spiral traces. As a result, the capacitance of the LC circuit increases, leading to a reduction in the resonance frequency. Although these sensors hold great potential for pressure measurements, one of the challenges lies in achieving a higher resonance frequency shift in response to the same amount of applied pressure, thereby enhancing their sensitivity. Strategies to improve sensitivity typically focus on modifying the polymeric dielectric layer or incorporating structured designs, such as cylindrical, cubical, angled, or pyramid-shaped configurations. However, these structured designs frequently encounter mechanical stability issues, limiting their effectiveness in improving sensitivity. One of the main challenges includes difficulty in the proper formation of the microstructures and providing sufficient elasticity with minimum hysteresis to ensure their return to the original form after pressure is released.

The present disclosure provides a pressure sensor with enhanced sensitivity and in vivo performance and a method of fabricating an ultra-sensitive wireless capacitive pressure sensor for biomedical applications using a structured nanocomposite dielectric layer. An inorganic compound is integrated into a polymer matrix and then fabricated into a micro-structured nanocomposite dielectric layer. The method involves fabricating a nanocomposite-based dielectric layer by integrating a dielectric compound into a polymer matrix. This approach provides optimized mechanical support to the structures, ensuring that more of them are properly formed. This unique advantage not only increases the sensitivity of the sensors to applied pressure but also strengthens the structures on the dielectric layer, preventing deformation after pressure is applied. Additionally, this method does not significantly change the dielectric constant of the polymer, thereby preventing sensitivity degradation.

The present disclosure provides enhanced sensitivity, enhanced pressure measurement, and minimized dielectric constant change. The sensitivity of the pressure sensor is enhanced because the nanocomposite-based dielectric layer in the pressure sensor provides robust mechanical support for the micro-structures, considerably boosting the sensor's sensitivity. The nanocomposite ensures that the structures are properly formed during the curing process of the dielectric mixture. Consequently, more structures are formed correctly and with greater strength, ensuring a uniform and optimal distance between the antenna layers. This approach increases sensitivity to applied pressure without introducing the complexity of new structure designs.

The nanocomposite-based dielectric material used in the wireless capacitive pressure sensor greatly improves the resolution of pressure measurements derived from the resonance frequency of the sensor at each pressure level. The inclusion of nanocomposites amplifies the shift in resonance frequency caused by applied pressure, allowing for more precise pressure extraction. This amplification results in more accurate pressure measurements, thereby enhancing the overall quality of data acquisition in wireless capacitive pressure sensors.

The method of fabrication of the pressure sensor has a minimal impact on the dielectric constant of the fabricated dielectric layer. The concentration of the compound integrated in the polymer matrix is below 1%, ensuring that the dielectric constant of the resulting dielectric layer remains largely unchanged and does not lead to a decrease in the initial resonance frequency of the sensor.

In some embodiments, biocompatible zinc oxide (ZnO) nanoparticles are incorporated into styrene-ethylene-butylene-styrene (SEBS) to fabricate a nanocomposite pyramid-structured dielectric layer used for fabricating pressure sensors. The incorporation of zinc oxide nanoparticles into the styrene-ethylene-butylene-styrene matrix results in mechanical strength and pressure sensitivity. Polydimethylsiloxane-parylene encapsulation ensures biocompatibility, and a multi-loop pickup coil extends the detection distance.

Other aspects of the disclosure will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an overview of the performance and in vivo application of an ultra-high sensitive nanocomposite-based wireless capacitive pressure sensors according to an embodiment of the present disclosure. a) Enhancement of the sensors' sensitivity by developing an elastomer nanocomposite dielectric layer with pyramid-like microstructure. b) Wireless measurement of the ICP variations using the capacitive pressure sensors fabricated with dielectric nanocomposites.

FIG. 2 illustrates the evaluation of pickup probes for contactless in vivo measurement of the pressure variations. a) Single-loop (left) and multi-loop (right) pickup probes modeled in HFSS. b) H-field plot on the multi-loop (top) and single-loop (bottom) pickup probe planes. c) Simulated S₁₁ results using single-loop and multi-loop pickup probes. d) Digital photograph of the fabricated pickup probes with two different designs. e) Comparison of the experimental performance of the fabricated pickup probes at various distances (0-10 mm) from the sensor. f) MATLAB-processed S₁₁ data at 8.4 mm distance from the sensors, measured with the multi-loop pickup probe.

FIG. 3 illustrates a fabrication process and characterization of the ZnO-based dielectric layers with varying concentrations of nanoparticles embedded in them. a) Schematic showing the process used for fabrication of the nanocomposite dielectric layers. b) FTIR spectra of the pure SEBS layer, ZnO NPs, and ZnO-SEBS nanocomposite layer. c) XRD patterns of a pure SEBS layer, ZnO NPs, and ZnO-SEBS nanocomposite layer. d-i) EDS mapping of the ZnO-SEBS dielectric layer at ZnO volumetric ratios of 0.1%, 0.3%, 0.5%, 0.7%, 1%, and 1.2%, respectively.

FIG. 4 illustrates the effect of ZnO nanoparticles on morphological uniformity of the pyramid structures. a-g) SEM images of ZnO-SEBS dielectric layers at ZnO volumetric ratios of 0%, 0.1%, 0.3%, 0.5%, 0.7%, 1%, and 1.2%, respectively. h) SEM image of the cross-section of a typical parylene-PDMS encapsulated sensor.

FIG. 5 illustrates the sensitivity analysis of pressure sensors made from dielectric layers with varying concentrations of ZnO nanoparticles. a) Measurement setup for detecting changes in the resonance frequency of the pressure sensors, due to applied pressure. b) Sensitivity of sensors made of nanocomposite dielectric layers containing different concentrations of ZnO. c) Resonance frequency shift under applied pressures of 0 and 100 mmHg for the sensors with pure SEBS dielectric layer and 0.7% ZnO-SEBS nanocomposite dielectric layer, before and after encapsulation. d) Resonance frequency behavior of encapsulated and non-encapsulated sensors with a pure SEBS dielectric layer and a 0.7% ZnO-SEBS nanocomposite dielectric layer under applied pressures ranging from 0 to 100 mmHg.

FIG. 6 illustrates the evaluation of cytotoxicity and in vivo functionality of the pressure sensors. a) Comparison of pH test paper strips incubated in three conditions: non-encapsulated pH paper immersed in aCSF, parylene-PDMS encapsulated pH paper immersed in aCSF, and non-encapsulated pH paper without any immersion in aCSF. b) Dimensional comparison of a representative encapsulated pressure sensor with a quarter coin. c, d) Cell viability test results on NIH-3T3 and GL261 cells, respectively, with results for each group shown in triplicate (one-way ANOVA, p<0.05). e) Resonance frequency shift of an implanted sensor in response to ICP change in a mouse, due to minor abdominal compression.

FIG. 7 illustrates H-field and E-field plots. a, b) H-field plot on the sensor plane when the multi-loop and single-loop pickup probes are used, respectively. c, d) E-field plot on the sensor plane when the multi-loop and single-loop pickup probes are used, respectively.

FIG. 8 illustrates a resonance frequency shift under applied pressure of 0 mmHg and 100 mmHg in the sensors made of 0.1% ZnO-SEBS nanocomposite dielectric layer.

FIG. 9 illustrates a resonance frequency shift under applied pressure of 0 mmHg and 100 mmHg in the sensors made of 0.3% ZnO-SEBS nanocomposite dielectric layer.

FIG. 10 illustrates a resonance frequency shift under applied pressure of 0 mmHg and 100 mmHg in the sensors made of 0.5% ZnO-SEBS nanocomposite dielectric layer.

FIG. 11 illustrates a resonance frequency shift under applied pressure of 0 mmHg and 100 mmHg in the sensors made of 1% ZnO-SEBS nanocomposite dielectric layer.

FIG. 12 illustrates a resonance frequency shift under applied pressure of 0 mmHg and 100 mmHg in the sensors made of 1.2% ZnO-SEBS nanocomposite dielectric layer.

FIG. 13 illustrates the effect of the number of loops in the multi-loop pickup probe on the measured return loss (−S₁₁). The return loss steadily increased up to 9 loops, then plateaued.

FIG. 14 illustrates the effect of embedded nanoparticles on the friction force between the dielectric layer and the PDMS-based mold during the peeling process to detach the dielectric layer from the soft mold.

FIG. 15 illustrates TEM images of ZnO NPs used for fabrication of SEBS-ZnO nanocomposites.

FIG. 16 illustrates cross-sectional SEM images. a) The height of a well-formed pyramid: about 41 μm. b) The base thickness of the dielectric layer: about 10 μm.

FIG. 17 illustrates a comparison of pH paper colors. a) Immersed in Sil-Poxy. b) In close proximity (~1 cm) to Sil-Poxy. c) Before any exposure to Sil-Poxy. Color variations confirmed the relatively mild acidic nature of Sil-Poxy and its vapors during the encapsulation process. These characteristics were rapidly transient and did not have a negative impact on performance of our sensors.

FIG. 18 the effect of parylene coating as a double protection layer to prolong sensors lifetime. a) PDMS encapsulated pH paper. b) Parylene-PDMS encapsulated pH paper. Both had an intentional pinhole in the PDMS layer and were immersed in aCSF. The arrow highlights the color change in the PDMS encapsulated pH paper, indicating solution penetration through PDMS hole, whereas the parylene coated sample remained unaffected.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The invention is capable of other embodiments and of being practiced or of being carried out in various ways.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifiers “about” or “approximately” used in connection with a quantity are inclusive of the stated value and have the meaning dictated by the context (for example, it includes at least the degree of error associated with the measurement of the quantity). These modifiers should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example, “about 1” may also mean from 0.5 to 1.4.

For the recitation of numeric ranges herein, each intervening number therebetween with the same degree of precision is contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated.

Definitions of chemical terms are described in more detail below. For purposes of this disclosure, the chemical elements are identified in accordance with the Periodic Table of the Elements, CAS version, Handbook of Chemistry and Physics, 104th Ed., inside cover, and specific functional groups are defined as described therein.

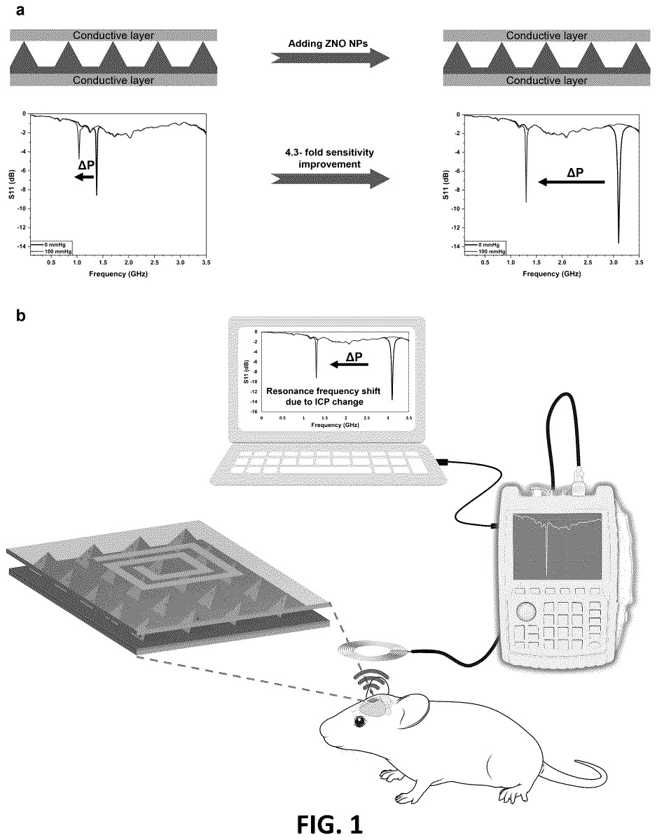

The present disclosure provides a system and method to address the mechanical stability challenges of structured dielectric layers and to enhance the sensitivity of capacitive pressure sensors by employing nanocomposite structures. With reference to FIG. 1 (at a), the present disclosure provides a pressure sensor 10 including a first conductive layer 14, a second conductive layer 18, and a dielectric layer 22 between the first conductive layer 14 and the second conductive layer 18. The first conductive layer 14 and the second conductive layer 18 include one or more spiral-shaped traces on a substrate. In one aspect, the first conductive layer 14 and the second conductive layer 18 include two spiral-shaped traces on a 100 μm thick polyimide substrate. The spiral traces include two turns, each with an initial length of 4 mm, a width of 0.4 mm, a 0.4 mm gap between the traces, and a thickness of 17.5 μm.

The dielectric layer 22 includes an inorganic compound that is integrated into a polymer matrix. The inorganic compound can comprise zinc oxide, silicon carbide, and the like. In an example, zinc oxide nanoparticles (ZnO NPs) that were integrated into a polymeric matrix, were added to SEBS polymer in various concentrations and fabricated as pyramid-shaped structures to provide the dielectric layer.

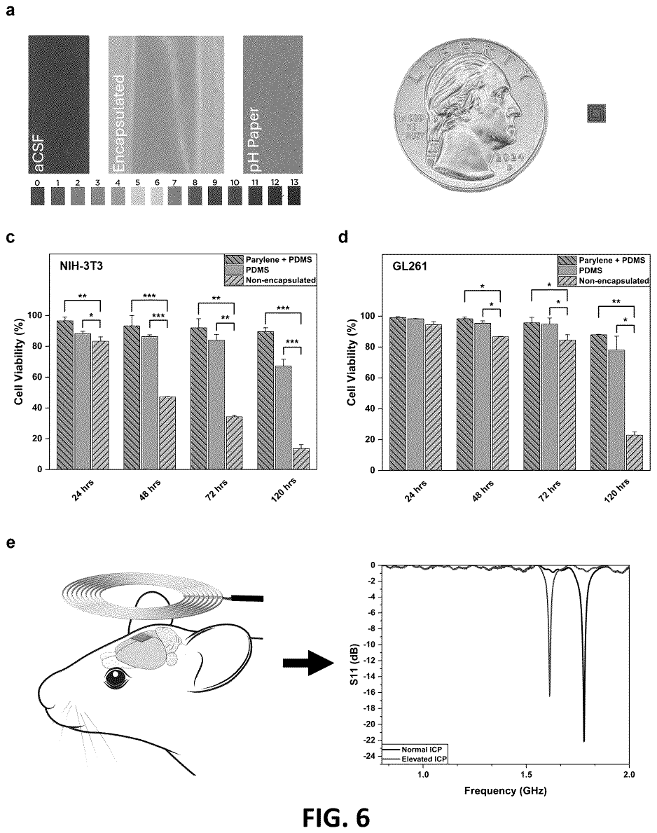

Additionally, the present disclosure provides a multi-loop pickup probe to maximize the reading distance utilizing a MATLAB code to detect resonance frequency at extended ranges. The sensors were encapsulated in PDMS and coated with parylene to improve biocompatibility, which was evaluated through the MTT assay. In vivo studies were conducted to assess the functionality of the implanted sensors in detecting ICP changes in the mouse brain (FIG. 1 (at b) and FIG. 6 (at e)). These results demonstrated the potential of these highly sensitive sensors for use in wearable and implantable bioelectronic devices and wireless pressure monitoring technology.

### I. PICKUP PROBE DESCRIPTION

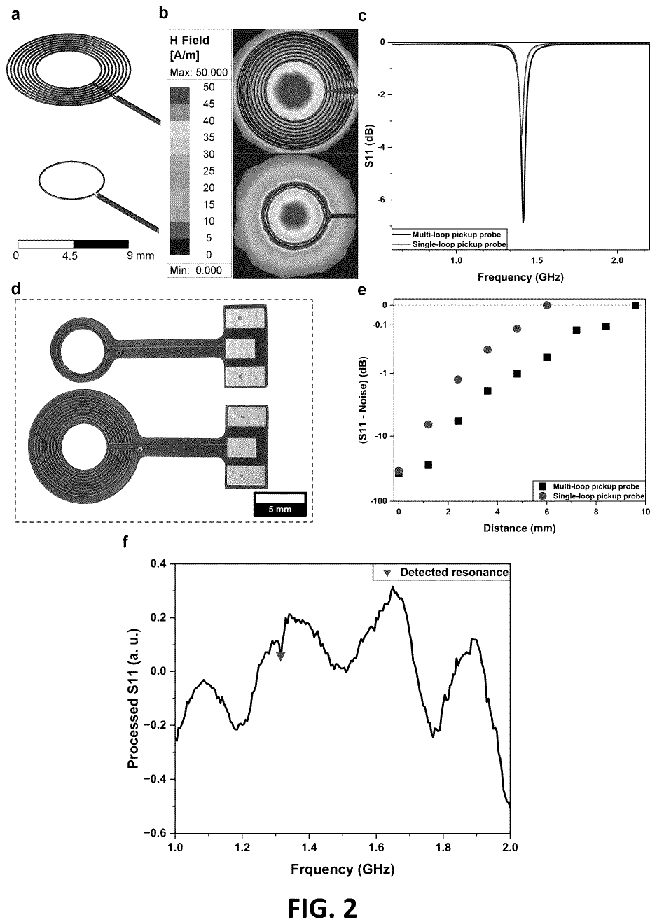

The configuration of the pickup probe in passive wireless capacitive pressure sensors played a role in determining the maximum distance at which resonance frequency data, and consequently pressure data, can be detected. The pickup probe was configured for maximizing the reading range of the sensors. Full-wave EM simulations were conducted using HFSS Electronics Desktop software. Simulations compared the effectiveness of the commonly used single-loop pickup probe with the multi-loop pickup probe described herein (FIG. 2 (at a)) in detecting the resonance frequency of capacitive pressure sensors.

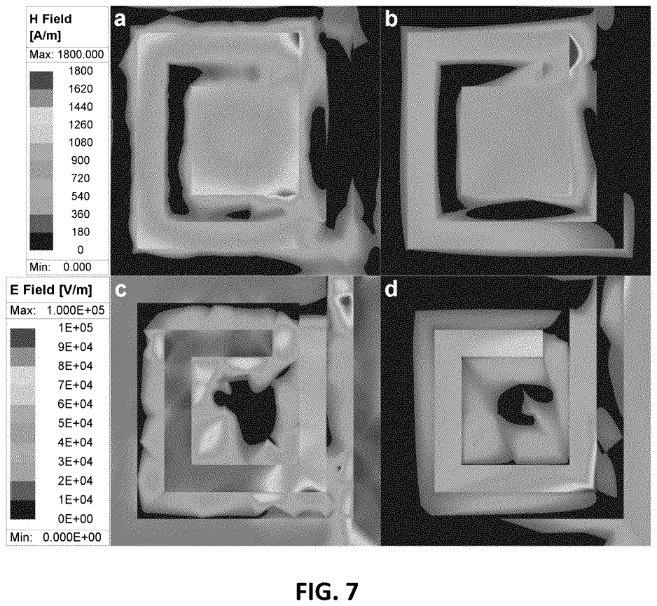

Capacitive pressure sensors' model with a defined SEBS dielectric layer were fabricated, with parameters aligned to practical dimensions to simulate real-world conditions for testing each probe. The magnetic fields (H-fields) at the plane of each probe were plotted, and it was found that the multi-loop pickup probe generated a stronger H-field (FIG. 2 (at b, top)) compared to the conventional single-loop probe (FIG. 2 (at b, bottom)) with an equal excitation power. This result indicated that the H-fields from each loop were constructively added at the center of the multi-loop pickup probe, resulting in an overall enhanced H-field strength. The plotted H-fields and E-fields on the sensor, corresponding to the use of either pickup probe, are presented in FIG. 7.

FIG. 2 (at c) illustrates the measured reflection coefficient (S₁₁) for both designs, showing a higher return loss (−S₁₁) of 6.72 dB at resonance frequency with the multi-loop pickup probe, compared to 3.55 dB at resonance frequency achieved with the single-loop pickup probe. These findings were further supported through experimental results using the fabricated single-loop and multi-loop pickup probes (FIG. 2 (at d)). As depicted in FIG. 2 (at e), the difference between the S₁₁ values and the noise level at the resonance frequency, measured by the multi-loop pickup probe, was consistently higher than those obtained with the single-loop probe at various distances from the sensors. The maximum detectable distance for the resonance signal was found to be 4.8 mm with the single-loop probe and 8.4 mm with the multi-loop probe.

Additionally, FIG. 2 (at f) presents the MATLAB-processed S₁₁ data obtained from the multi-loop probe at a distance of 8.4 mm, accurately detecting the resonance frequency of the sensors at this separation. Collectively, these results demonstrated the superior performance of the multi-loop pickup probe compared to the traditional single-loop design, confirming its effectiveness in extending the detection range.

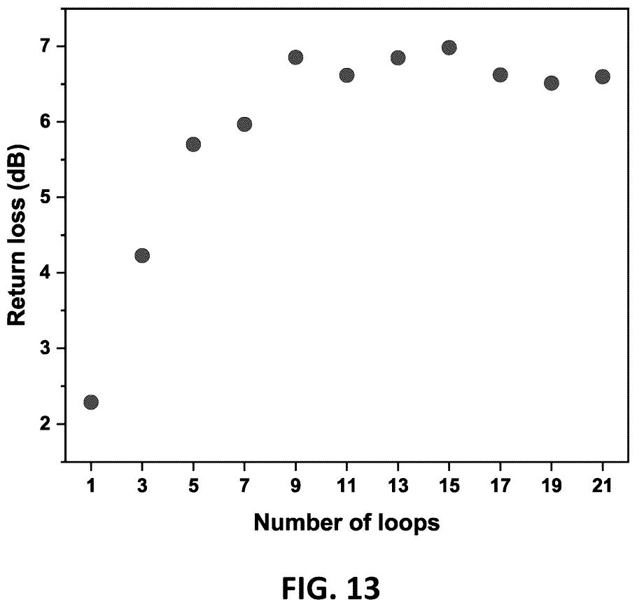

Effect of the number of the pickup probe loops on its resonance frequency detection range was further investigated through HFSS simulations, as shown in FIG. 13. These simulations assessed the impact of increasing the number of loops on the return loss to determine the optimal probe design. As illustrated in FIG. 13, a multi-loop pickup probe with nine loops was found to be optimal.

### II. MATERIALS CHARACTERIZATION

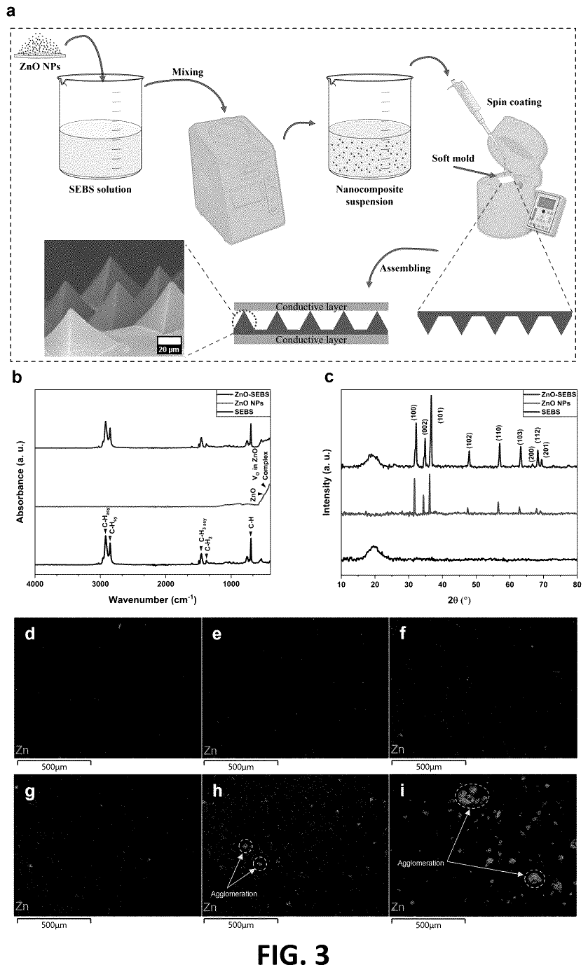

FTIR Characterization. FIG. 3 (at a) illustrates the fabrication process of the ZnO-SEBS nanocomposite dielectric layer, with detailed steps outlined below. FIG. 3 (at b) presents the Fourier transform infrared (FTIR) spectra of pure SEBS, ZnO NPs, and ZnO-SEBS nanocomposite in absorbance mode. In the SEBS spectrum, the absorbance peak around 690 cm⁻¹ corresponded to the out-of-plane bending of the CH bonds in the aromatic ring. The absorbance in 1378 cm⁻¹ was related to the deformation vibration of CH bonds in the CH₂ groups. The peak around 1450 cm⁻¹ stood for the asymmetric stretching of the CH bond in the CH₃ groups of the ethylene-butylene blocks in SEBS molecular structure. Finally, the peaks at 2845 cm⁻¹ and 2916 cm⁻¹ belonged to symmetric and asymmetric vibrations of the CH bonds in the CH₂ parts. The FTIR spectrum of ZnO NPs showed no peak in the range of 1000 to 4000 cm⁻¹, indicating the absence of carboxylate and hydroxyl impurities. This suggested that the nanoparticles were vacuum-annealed after synthesis. Additionally, two intense peaks were present at 437 cm⁻¹ and 496 cm⁻¹, which were attributed to the E2 mode of hexagonal ZnO and oxygen deficiency (V_{O}) in the ZnO structure, respectively. In the ZnO-SEBS nanocomposite spectrum, all characteristic peaks of both ZnO and SEBS were observable, confirming the successful formation of the desired nanocomposite.

XRD Characterization. FIG. 3 (at c) depicts the X-ray diffraction (XRD) patterns of pure SEBS, ZnO NPs, and ZnO-SEBS nanocomposite. The XRD pattern of pure SEBS suggested an amorphous structure, with a broad peak at 2θ=15-25°, that could be related to the styrene phase in the SEBS molecular structure. All peaks corresponding to the XRD pattern of ZnO were labeled and shown in FIG. 3 (at c), confirming the formation of the wurtzite structure of zinc oxide. In the XRD pattern of the ZnO-SEBS nanocomposite, all the peaks of the ZnO NPs and the amorphous SEBS peak were present, suggesting the successful formation of the ZnO-SEBS nanocomposite without any additional peaks from potential impurities.

FWHM analysis of the XRD peaks in ZnO NPs and ZnO-SEBS spectra (Table 1) revealed that the crystalline peaks of ZnO broadened slightly, when the nanoparticles were embedded in the SEBS matrix. Note that peak intensity units are arbitrary in FIG. 3.

TABLE 1
Calculated FWHM Values for Major ZnO Diffraction
Peaks in ZnO NPs and ZnO-SEBS.

| | | FWHM | FWHM |
|---|---|---|---|
| Peaks Center | Plane Group | ZnO-NPs | ZnO-SEBS |
| 31.7 | (100) | 0.088 | 0.164 |
| 34.4 | (002) | 0.0691 | 0.119 |
| 36.2 | (101) | 0.0676 | 0.223 |

EDS Characterization. FIG. 3 (at d-i) exhibits the energy-dispersive X-ray spectroscopy (EDS) mapping data of the ZnO-SEBS nanocomposites with different concentrations. As shown in FIG. 3 (at d-g), ZnO NPs were uniformly distributed in the nanocomposite layers at concentrations below 0.7% v/v without significant agglomeration. However, at higher concentrations, a noticeable aggregation of ZnO NPs appeared in the nanocomposite.

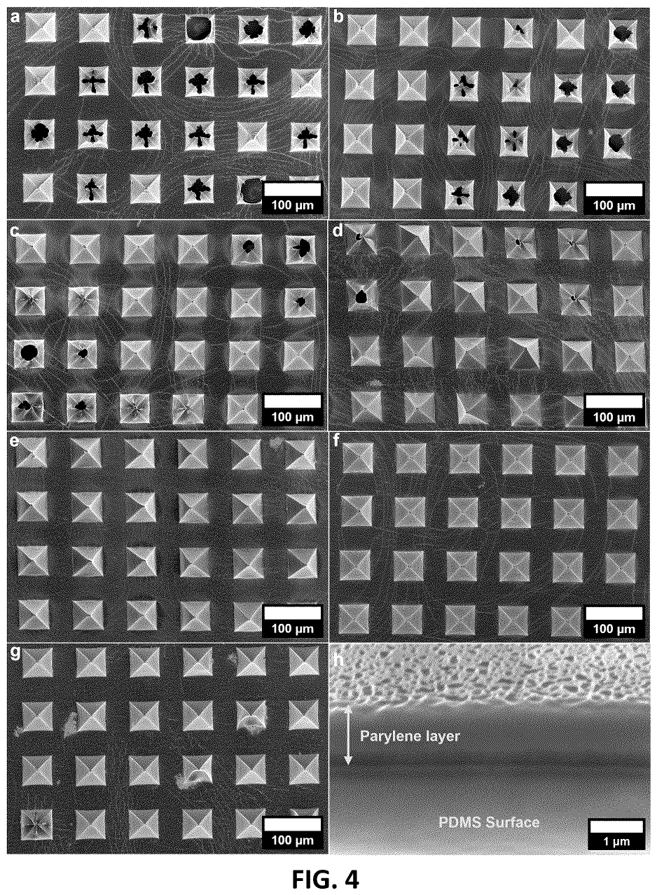

SEM Characterization. FIG. 4 (at a-g) present the scanning electron microscopy (SEM) images of the dielectric layers with different concentrations of ZnO NPs, demonstrating an increase in well-formed pyramid-shaped structures within the same field of view as the concentration of ZnO NPs in the SEBS matrix increased. This increase was attributed to the greater mechanical support provided by the presence of ZnO NPs within the SEBS matrix. This implied that the high mechanical strength of ZnO NPs, in the GPa scale, significantly improved the mechanical strength of the nanocomposite compared to the pure SEBS dielectric layer with mechanical strength in the order of MPa. Even at lower concentrations, this reinforcement helped the pyramid-shaped structures withstand fabrication stress, leading to better formation of intact pyramids and, consequently, higher sensor sensitivity. The sensors were encapsulated with a parylene-PDMS layer to prevent fluid diffusion into them and improve their biocompatibility. FIG. 4 (at h) presents the cross-section view of a typical parylene-PDMS encapsulated sensor confirming the successful formation of the 2 μm parylene on the surface of the PDMS layer.

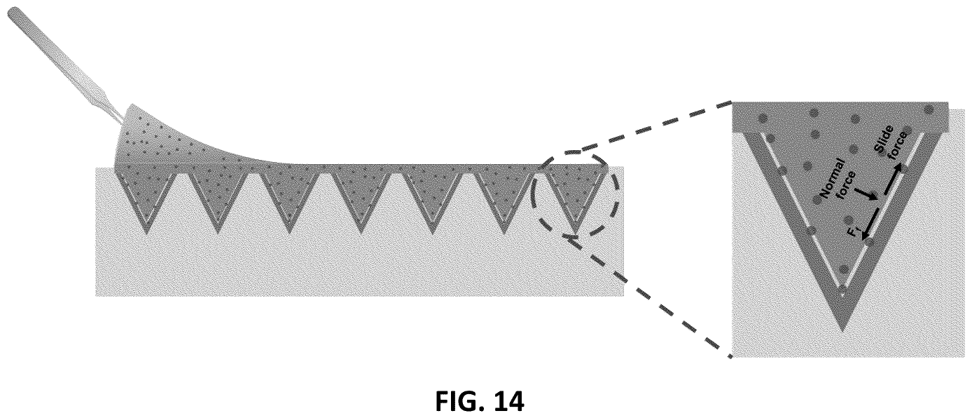

The dielectric fabrication procedure induces stress in the dielectric layer, with the most significant stress arising from friction and peeling forces during the demolding process. Specifically, as the dielectric layer is peeled from the mold, stresses arise from the peeling force, friction, and intermolecular adhesion due to polymer chains bridging across the interface between the dielectric layer and the PDMS mold. These polymer chains contribute to adhesive forces that can resist separation, increasing stress on the dielectric layer (FIG. 14).

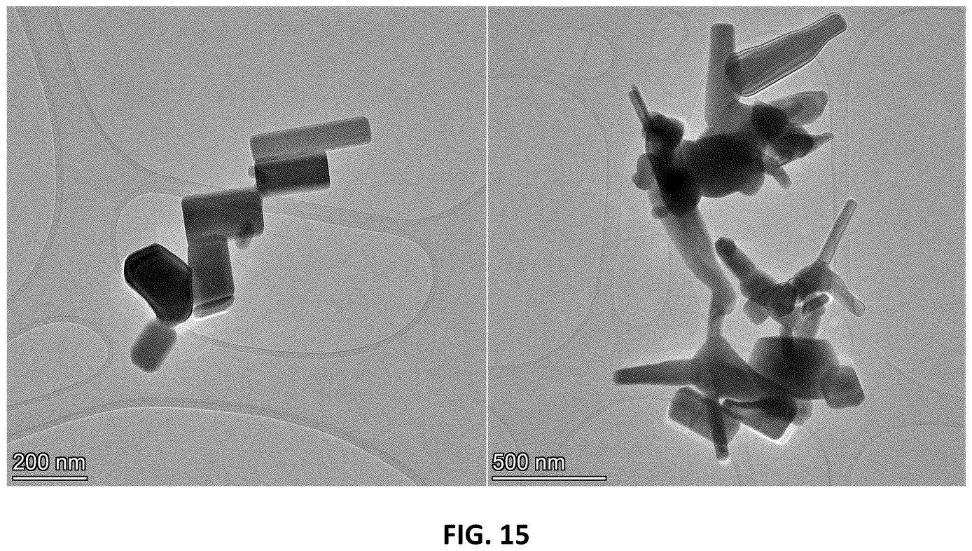

Other factors influencing the reinforcement effect of nanoparticles were their morphology and crystal structure. Nanoparticles with higher aspect ratio and large surface areas, such as rod-shaped ZnO nanoparticles used herein (FIG. 15) have high interfacial adhesion to the polymer matrix and were effective in stress transfer, enhancing the reinforcement effect. A more uniform distribution of these rod-shaped nanoparticles was important to further enhance the mechanical strength of the composite, and as a result, the sensitivity and life-time of the sensors.

### III. ELECTRICAL MEASUREMENT

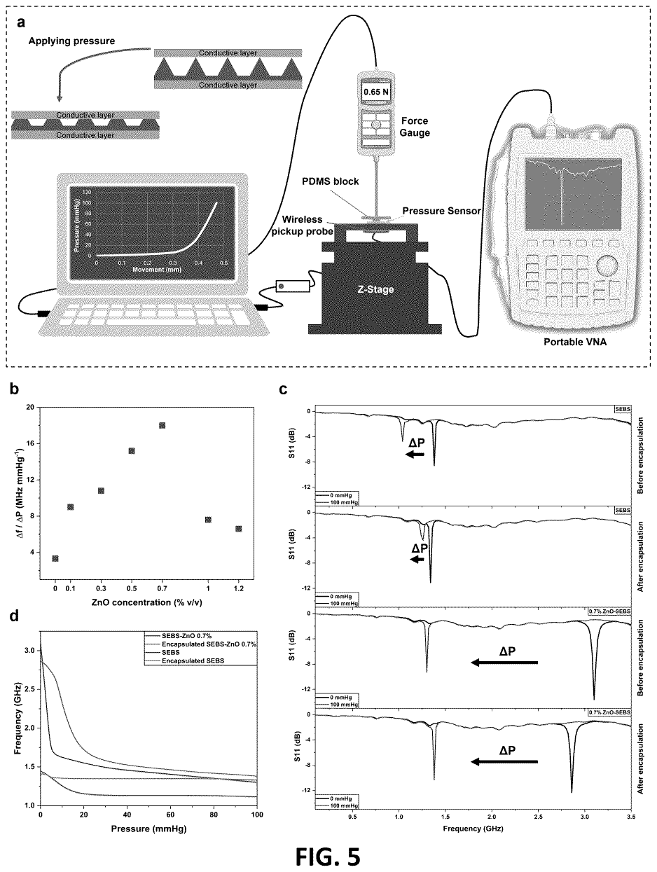

The sensitivity of each sensor was measured using the setup illustrated in FIG. 5 (at a), with the detailed measurement process outlined in the Experimental Section. The sensitivity of each sensor in response to applied pressure is defined as follows:

$$Sensitivity = \vert \frac{Δ f}{Δ p} \vert \qquad ( 1 )$$

where Δf represents the change in resonance frequency (in MHz) due to applied pressure, and ΔP is the variation in pressure (in mmHg).

FIG. 5 (at b) presents the impact of the nanocomposite dielectric layers at different concentrations on the sensitivity of the fabricated pressure sensors, calculated for pressure changes ranging from 0-100 mmHg. The sensors exhibited a sensitivity of 3.31 MHz mmHg⁻¹ when pure SEBS was used as the dielectric material. However, incorporating ZnO NPs into the SEBS layer significantly enhanced sensitivity, rising to 9, 10.8, 15.2, and 18 MHz mmHg⁻¹ at ZnO concentrations of 0.1%, 0.3%, 0.5%, and 0.7% v/v, respectively. Further increases in ZnO concentration degraded the sensitivity, which dropped to 7.6 and 6.6 MHz mmHg⁻¹ at ZnO concentrations of 1% and 1.2%, respectively. This was consistent with the EDS maps, which indicated that beyond the optimal concentration of 0.7%, ZnO nanoparticles began to aggregate, forming barriers that hindered the full compression of the pyramidal structures and thereby inhibited the complete shift of the resonance frequency to lower values. The top row of FIG. 5 (at c) shows detailed S₁₁ data for the sensors using a dielectric layer composed of pure SEBS, under pressures of 0 mmHg and 100 mmHg. The initial resonance frequency of the sensors with this dielectric layer was 1.44 GHZ, which was lower than the sensors employing 0.7% ZnO-SEBS nanocomposite dielectric layer, resonating at 3.1 GHz (FIG. 5 (at c, third row)). This increase in initial resonance frequency after adding ZnO NPs to pure SEBE was attributed to the improved structural integrity of the pyramidal shapes due to the mechanical support provided by ZnO NPs, which increased the distance between the conductive layers, reduced the initial capacitance, and consequently raised the initial resonance frequency and sensitivity. PDMS encapsulation and parylene coating of the sensors with the SEBS dielectric layer significantly reduced their sensitivity to 0.78 MHz mmHg⁻¹, as depicted in the second row of FIG. 5 (at c, second row). However, the presence of the ZnO NPs partially mitigated this negative effect, and the sensitivity of the sensor with 0.7% ZnO-SEBS nanocomposite dielectric layer remained high at 14.8 MHz mmHg⁻¹ (FIG. 5 (at c, fourth row)).

FIG. 5 (at d) demonstrated the relationship between resonance frequency change and applied pressure for sensors made of pure SEBS and 0.7% ZnO-SEBS nanocomposite dielectric layers, both before and after encapsulation. Groups of sensors composed of a 0.7% ZnO-SEBS nanocomposite as their dielectric layer had a high linear sensitivity of 45 MHz mmHg⁻¹ up to 25 mmHg and 18 MHz Hg⁻¹ up to 100 mmHg.

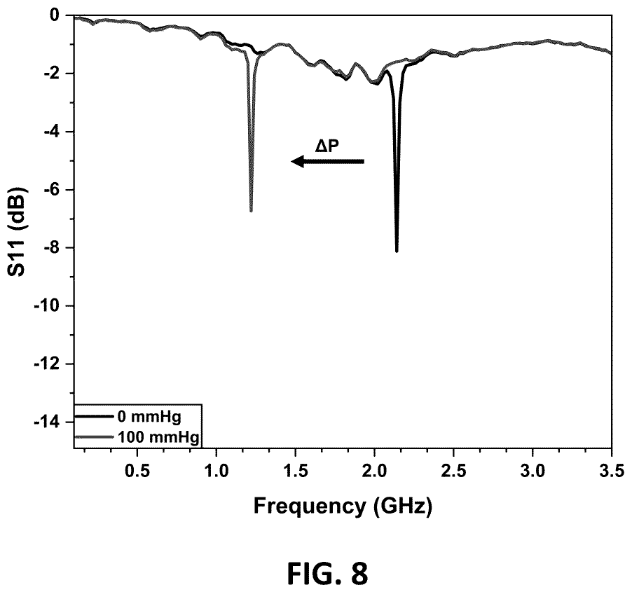

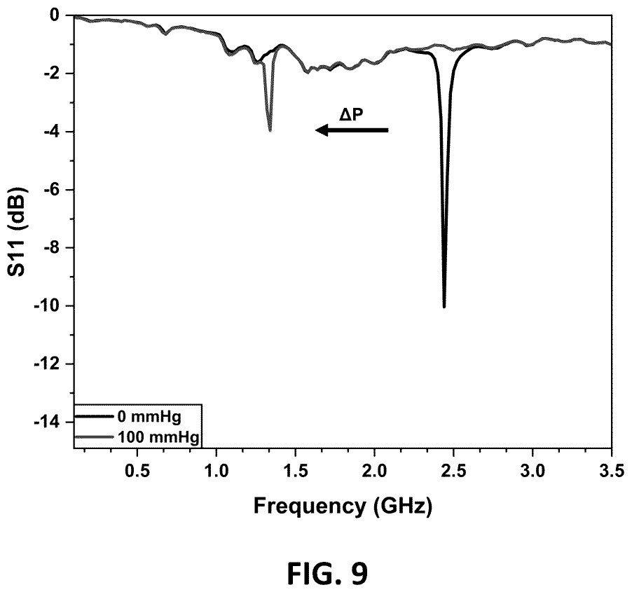

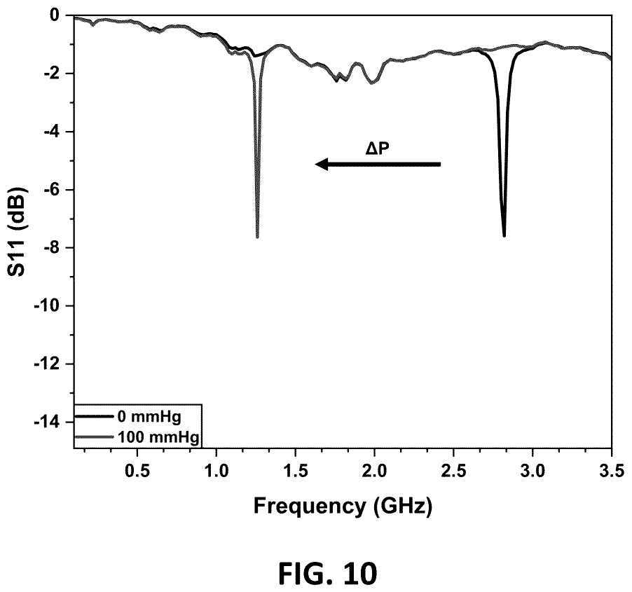

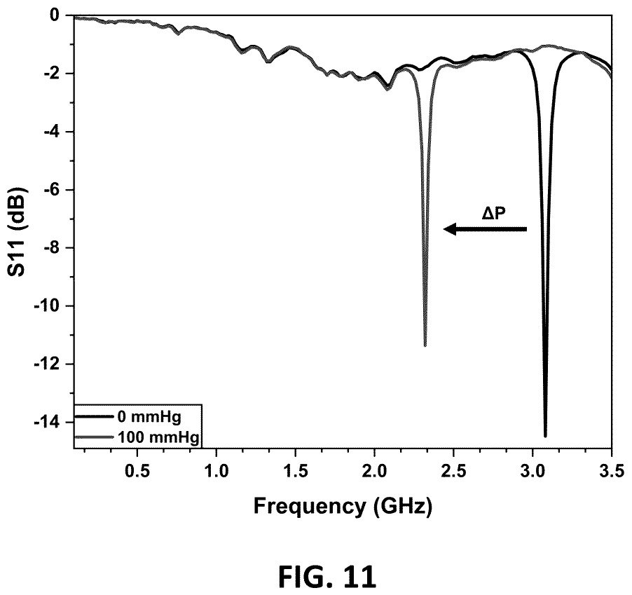

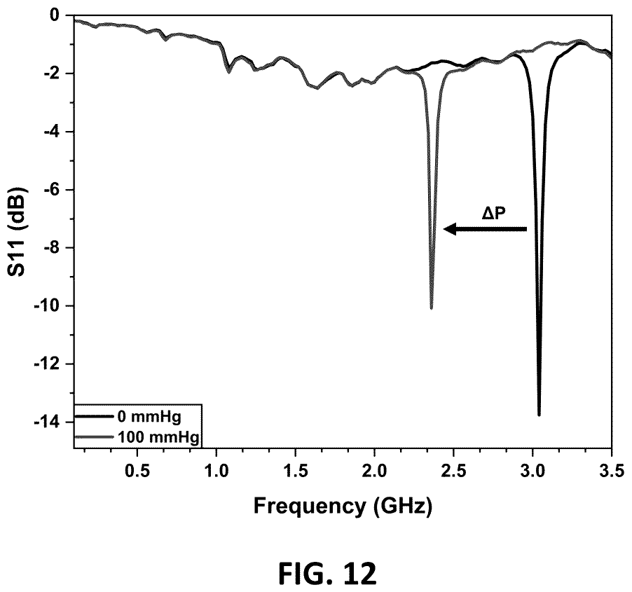

Notably, the sensors with the nanocomposite dielectric layer demonstrated extended sensitivity at higher pressures compared to the pure SEBS sensors, indicating an additional advantage of using a nanocomposite dielectric layer in wireless capacitive pressure sensors. Detailed frequency measurements and analysis for the fabricated sensors with various ZnO concentrations in the dielectric layer can be found in FIGS. 8-12.

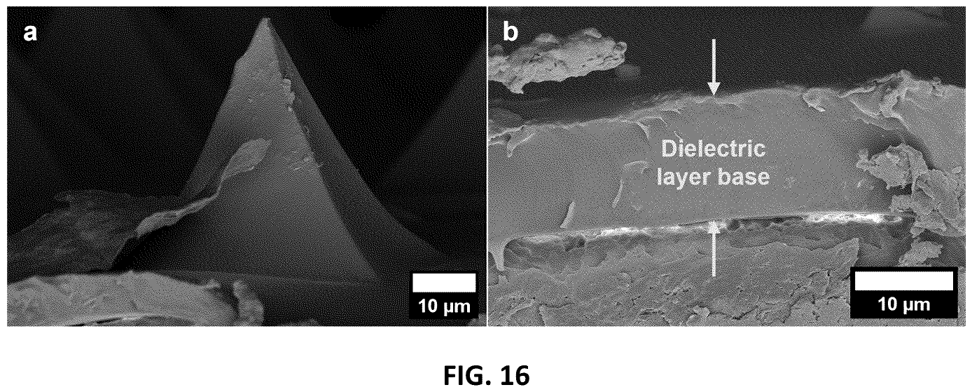

The maximum initial resonance frequency was observed when a 0.7% or higher ZnO-SEBS dielectric layer was used, indicating the greatest distance between the conductive layers. This distance was estimated to be 51 μm, calculated by summing the average base thickness of the dielectric layer (~10 μm) with the height of the pyramidal structures (~41 μm), as measured using cross-sectional SEM images at several locations (FIG. 16).

### IV. EVALUATION OF CYTOTOXICITY AND IN VIVO PERFORMANCE

Sealing Strength Test. One purpose of encapsulating the sensors is to prevent fluid diffusion into the system. The effectiveness of the encapsulation method was evaluated by investigating sealing strength using pH test paper (Hydrion®) and artificial cerebrospinal fluid (aCSF) prior to the cytotoxicity test. FIG. 6 (at a) compares the color changes of different types of pH paper strips (i.e., non-encapsulated paper, parylene-PDMS encapsulated paper), before and after immersion in aCSF. As depicted in FIG. 6 (at a), the aCSF solution was moderately basic, turning the color of the non-encapsulated paper green upon immersion. The color of the encapsulated pH paper indicated a more acidic environment compared to the non-immersed paper. These results demonstrated that the parylene-PDMS encapsulation sealed the sensors effectively.

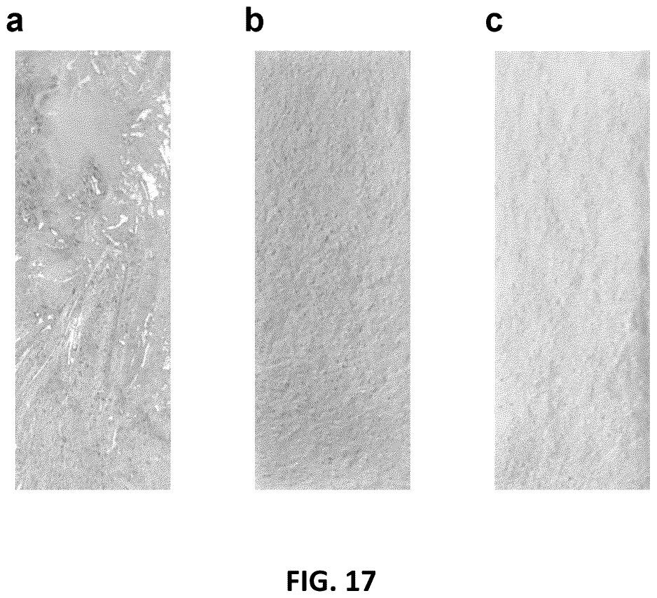

Sil-Poxy and its vapors are mildly acidic, as verified by pH evaluations shown in FIG. 17. Therefore, to prevent any potential negative impacts, the devices were encapsulated under a constant air flow inside the fume hood. Additionally, the encapsulated sensors were exposed to a 30 mTorr vacuum to ensure that no air or acidic vapors were trapped in the encapsulation layer, because their presence could disrupt the uniform pressure distribution in sensors and diminish their performance.

Cytotoxicity Test. The MTT (methyltetrazolium salt) assay was employed to evaluate the cytotoxicity of the three fabricated biosensors. This quantitative assay is an indicator of cell viability following exposure to the various sensor encapsulations and incubation periods. The results of the cytotoxicity tests conducted on parylene-PDMS encapsulated sensors, PDMS-encapsulated sensors, and non-encapsulated sensors (with a dimensional comparison to a quarter coin shown in FIG. 6 (at b)) using NIH-3T3 (murine fibroblast) and GL261 (murine brain tumor) cell lines are presented in FIG. 6 (at c) and FIG. 6 (at d), respectively. These results were consistent across both cell lines, showing that after 24 hours, cell viability was highest in the parylene-PDMS encapsulated group, followed by the PDMS-encapsulated group and then the non-encapsulated sensors. Statistically significant differences (P<0.05) calculated for the time points starting from 48 hours onward between these groups were confirmed using one-way analysis of variance (ANOVA), indicating that the parylene coating and encapsulation maintained the cell viability. Comparing the viability results with the control groups indicated that the parylene-PDMS encapsulated sensors maintained superior viability compared to PDMS-encapsulated sensors (P<0.05), while non-encapsulated sensors showed higher cytotoxicity (P<0.05) in both cell lines, particularly after 72 and 120 hours of incubation. These results align with existing literature, which demonstrated that both PDMS and parylene are biocompatible materials widely used for encapsulation. Coating PDMS-encapsulated sensors with parylene compensated for unavoidable fabrication defects, such as micro-holes and pinholes within the encapsulation layer, as parylene coating is a gas-based deposition method that creates a uniform coating across the entire surface of the sensors. These combined properties of PDMS and parylene enhanced the biocompatibility of the sensors, as demonstrated by the results in FIG. 6 (at c-d).

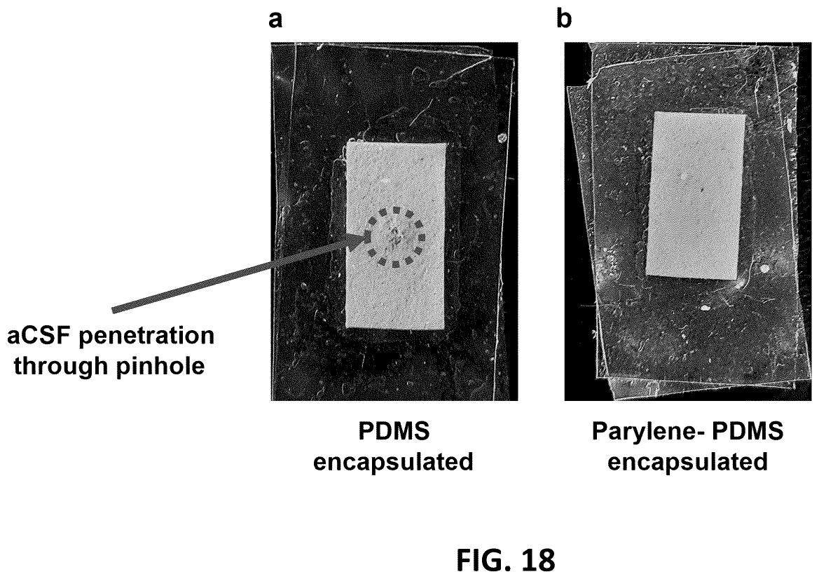

The effectiveness of parylene coating in mitigating fabrication defects was assessed. Two pH papers were encapsulated with PDMS, each with an intentional pinhole. One sample received parylene coating after the defect was introduced, while the other remained uncoated. Both samples were then immersed in aCSF. The results showed that the parylene coating effectively sealed the defect, whereas a visible color change was observed at the defect site in the uncoated sample (FIG. 18).

In Vivo Study. To further validate the functionality of the fabricated pressure sensors for monitoring internal pressures, such as intracranial pressure in the brain, in vivo studies were conducted on C57BL/6 mice. To implant the sensors on the mouse brain, the dimensions of the sensors were minimized by trimming the excess polyimide substrate surrounding the antenna traces. The sensors were encapsulated in parylene-coated PDMS, preparing them for implantation in the mouse brain. The pickup probe was placed in contact with the scalp on the implantation site, and the sensor S₁₁ factor was measured using a vector network analyzer. As shown in FIG. 6 (at e), the initial resonance frequency of the sensor after implantation dropped to 1.78 GHZ, reflecting the baseline ICP. After applying minor abdominal pressure with the index finger (Valsalva maneuver) for a short period of time (1-2 seconds), the resonance frequency decreased to 1.61 GHz due to the ICP increase. These results verified the functionality of the implanted pressure sensor in ICP measurement.

### V. EXAMPLES

Numerical Simulations: Numerical simulations were conducted using HFSS Electronics Desktop (ANSYS, Inc., Canonsburg, PA, USA) to compare the performance of two different pickup probes in detecting the resonance frequency of the capacitive pressure sensor. The sensors featured a dielectric layer with a dielectric constant of 3 and a thickness of 100 μm, simulating the properties of the SEBS dielectric layer. The created dielectric layer was positioned between two spiral-shaped traces on a 100 μm thick polyimide substrate. The spiral traces consisted of two turns, each with an initial length of 4 mm, a width of 0.4 mm, a 0.4 mm gap between the traces, and a thickness of 17.5 μm. The simulated pickup probes for performance comparison included a multi-loop probe consisting of nine concentric loops with a 0.15 mm gap between each loop, the smallest having a radius of 2.74 mm, and a single-loop probe with a similar radius. Both probes were modeled with a trace width of 0.15 mm and connected to a coaxial cable for signal transmission using 1 watt excitation. The probes were positioned 2.4 mm from the sensors to assess their effectiveness in detecting the resonance frequency of the sensors.

Fabrication of Pickup Probes and Spiral Traces: The printed circuit boards (PCBs) for the spiral traces and pickup probes were designed using KiCad PCB design software, following the parameters outlined above. The spiral traces were printed on a flexible 14 mm×14 mm polyimide substrate, with a substrate thickness of 100 μm and a trace thickness of 17.5 μm. In contrast, the pickup probes were fabricated on a rigid FR4 substrate, which had a substrate thickness of 600 μm and a trace thickness of 35 μm. All PCB designs were sent to PCBWay for fabrication.

Fabrication of Soft Mold: A structured soft mold with inverted pyramid-shaped cavities was fabricated using a method previously introduced by S. C. B. Mannsfeld et al. (Nature Materials, 2010, 9, 859-864). In summary, the procedure began with patterning a (100) silicon wafer through photolithography, followed by etching with a KOH solution. The indentations created on the silicon were then replicated in PDMS through a two-step molding process, first on the silicon and then on the replicated PDMS.

Preparation of Dielectric Layer: A 75 mg ml⁻¹ SEBS solution was prepared by dissolving the required amount of SEBS (H1053 grade, AsaheiKASEI) in dichloromethane (Sigma-Aldrich). The solution was subjected to vigorous stirring for 6 hours. Following this, ZnO NPs (Thermo Scientific Chemicals) were dispersed into the SEBS solution at volumetric concentrations of 0.1%, 0.3%, 0.5%, 0.7%, 1%, and 1.2% using a speed mixer (FlackTek™) at 300 rpm for 2.5 minutes. The resulting mixtures were then spin-coated onto a prefabricated soft mold with pyramid-shaped cavities using a spin coater (WS-650-23NPPB, Laurell Technologies Corporation) at 2000 rpm and an acceleration of 500 rpm s⁻¹ for 60 seconds, forming a pyramid-structured nanocomposite dielectric layer. Afterward, the dielectric layer was demolded and placed on one electrode, while the other electrode was positioned on top, and the sandwich structure was bonded by heating on a hot plate.

Encapsulation Procedure: The sensors were encapsulated using PDMS. A mixture of Sylgard™ 184, with a 10:1 ratio of elastomer base to curing agent, was prepared using a speed mixer at 2500 rpm for 5 minutes, followed by 90 minutes of degassing under vacuum. The mixture was then spin-coated onto microscope slides at 500 rpm with an acceleration of 100 rpm s⁻¹ for 90 seconds. Subsequently, the PDMS-coated slides were degassed under vacuum for 2 hours and cured in a vacuum oven (Isotemp, Fisherbrand™) for 18 hours at 70° C. After curing, the PDMS layers were demolded, and the sensors were positioned on one cured PDMS layer. A second PDMS layer was bonded to the first using a thin layer of Sil-Poxy adhesive around the perimeter of the sensors. The adhesive was allowed to cure at room temperature for 24 hours. Finally, the encapsulated sensors were coated with a 2 μm layer of parylene to ensure an effective seal. To achieve a uniform coating with the desired thickness, 1 gram of dimer C was loaded into a SCS Labcoater® 2 (PDS 2010), with a growth rate of 2 μm per gram.

Materials Characterization: Pure SEBS dielectric layer, ZnO NPs, and ZnO-SEBS nanocomposite dielectric layer were characterized using FTIR spectroscopy and XRD. FTIR measurements were performed in absorbance mode using a pike diamond ATR accessory in the wavenumber range of 400 cm⁻¹ to 5000 cm⁻¹, employing a Perkin Elmer Frontier FTIR spectrophotometer. XRD was carried out using a high-resolution (θ/θ) Rigaku SmartLab X-ray Diffractometer in the 2θ range of 10 to 80 degrees, utilizing Cu Kα radiation (λ=1.54178 Å). OriginPro software was used for curve fitting and analyzing the FTIR and XRD patterns to measure the required parameters.

The distribution of ZnO NPs in the SEBS matrix was examined via EDS mapping, using an Oxford Instruments X-Max detector. The pyramid-shaped structures on the surface of spin-coated nanocomposite dielectric layers were studied utilizing a field emission scanning electron microscope (FESEM-Auriga-Zeiss) at an accelerating voltage of 5 kV.

A parylene-PDMS encapsulated sensor was milled using a focused ion beam (Nova 200 NanoLab) with a 30 kV Ga beam for cross-sectional SEM imaging and thickness measurements of the encapsulation layers. Prior to milling, a Pt gas injection system was employed for in-situ deposition of 500 nm and 1 μm Pt layers using the electron beam and ion beam, respectively, at the top of desired location for milling. These steps were taken to minimize damage to the surface of the sample during the milling process and obtain clear cross-sectional images.

Resonance Frequency Measurement Setup: A high-resolution force gauge (M5-500-Mark-10) was synchronized with a Z-stage (Newmark Systems, Inc.) using LabVIEW (National Instruments) software for applying and real-time measuring pressure on the sensors. The sensors were positioned on a 1.2 mm thick microscope slide, placed 1 cm above the surface of the Z-stage, with the pickup probe attached to the opposite side of the slide. The pickup probe was connected to a vector network analyzer (FieldFox N9912C, Keysight Technologies, USA) to establish electromagnetic coupling with the sensor and to measure the S₁₁ parameter at each pressure point. In all experiments, a 7 mm×7 mm×1 mm PDMS block was placed on top of the sensors to ensure even pressure distribution. A custom MATLAB R2019b (MathWorks Inc.) code was developed to facilitate the detection of the resonance frequency of the sensors by detrending the S₁₁ data and identifying the deepest valley in the processed results.

Cytotoxicity Assay: The cytotoxicity of parylene-PDMS encapsulated sensors, PDMS-encapsulated sensors, and non-encapsulated sensors were evaluated using NIH-3T3 (murine fibroblast) and GL261 (murine brain tumor) cell lines in different timelines (24, 48, 72 and 120 hours). All cell lines were confirmed to be free from mycoplasma contamination. The cells were cultured in DMEM media (Fisher Scientific) supplemented with 10% (v/v) fetal bovine serum (FBS) and 1% penicillin-streptomycin and regularly maintained as monolayers in T-75 cm² culture flasks within a humidified incubator (PHCbi Cell IQ™ CO₂ Incubator) at 37° C., with 95% air and 5% CO₂. Once the cells reached 80% confluence, they were passaged to prepare for the experiments. A colorimetric MTT assay was employed to assess cell viability. For this, all samples were sterilized by soaking in 70% ethanol for 15 minutes, followed by a 2-hour drying process under a biosafety cabinet at room temperature. Then, U87 and GL261 cells were seeded in 6-well plates (100 k cells/well) and each group of samples was placed in triplicates with considering control groups for each subgroup (n=3). The samples were divided into four subgroups dedicated to each time point. At the end of time points, 200 μl of MTT solution (5 mg ml⁻¹ in PBS) was added to each well of the plates and were incubated for 4 hours at 37° C. Then, the medium was replaced with 1.2 ml of dimethyl sulfoxide (DMSO, VWR®) for 20 minutes and the absorbance was determined at 570 nm using HT1 plate reader (Biotek, USA). Cell viability was calculated using the following equation:

$$Cell viability = \frac{OD Samples}{OD Controls} \times 100 \qquad ( 2 )$$

where OD represents optical density. Statistical analyses were performed using SigmaPlot 12.0 (Systat Software, San Jose, CA, USA).

In Vivo Study: All animal studies were conducted according to the animal protocol approved by the Institutional Animal Care and Use Committee (IACUC) at Arizona State University (protocol No. 23-1981R). For this study, a healthy adult male C57BL/6 mouse was anesthetized with isoflurane in the anesthesia chamber and placed on a stereotactic surgery frame (Stoelting Co.), with the head secured by ear bars. A 1 cm incision was performed on the skull to reveal the coronal and sagittal sutures under a dissection microscope. The bregma was identified, and a 5×5 mm² square bone flap was carefully drilled with precision, centered 3 mm anterior and 3 mm lateral to the bregma. Then the bone flap was removed, and a sterilized 4×4 mm² parylene-PDMS encapsulated sensor was placed on the brain, ensuring the device was aligned 2 mm anterior and 2 mm lateral to the bregma. The bone flap was then reattached with surgical glue, and the skin incision was sealed using veterinary glue. After surgery, the fabricated multi-loop pickup probe, connected to the vector network analyzer, was positioned directly on the implantation site to measure ICP.

What is claimed is:

1. A pressure sensor comprising:
a first inductive conductor;
a second inductive conductor; and
a dielectric layer positioned between the first inductive conductor and the second inductive conductor, wherein the dielectric layer includes a plurality of micro-structured elastomers comprising a mixture of organic and inorganic polymers along with an inorganic compound.

2. The pressure sensor according to claim 1, wherein the inorganic compound comprises one or more materials selected from metal oxides, metal nanoparticles, metal oxide nanoparticles, ceramics, ionic compounds, or other functional fillers capable of forming a composite with the plurality of micro-structured elastomers.

3. The pressure sensor according to claim 2, wherein the metal oxide nanoparticle comprises zinc oxide nanoparticles.

4. The pressure sensor according to claim 3, wherein the zinc oxide nanoparticles include a concentration of between 0.1% v/v and 1.2% v/v.

5. The pressure sensor according to claim 4, wherein the zinc oxide nanoparticles include a concentration of 0.7% v/v.

6. The pressure sensor according to claim 1, further comprising an elastomer layer configured to encapsulate the first inductive conductor, the second inductive conductor, and the dielectric layer.

7. The pressure sensor according to claim 6, wherein the elastomer layer comprises one or more elastomers selected from silicone-based, styrenic block copolymers, or other polymeric elastomers, including but not limited to polydimethylsiloxane (PDMS), styrene-butadiene-styrene (SBS), and styrene-ethylene-butylene-styrene (SEBS).

8. The pressure sensor according to claim 7, further comprising a coating including parylene, and wherein the coating is applied to the elastomer layer.

9. The pressure sensor according to claim 1, wherein the pressure sensor exhibits a sensitivity between 3 MHz mmHg⁻¹ and 18 MHz mmHg⁻¹.

10. The pressure sensor according to claim 1, wherein the first inductive conductor and the second inductive conductor comprise a spiral-shaped trace on a polyimide substrate.

11. The pressure sensor according to claim 1, wherein the dielectric layer comprises a dielectric constant of 3 and a thickness of 100 μm.

12. The pressure sensor according to claim 1, wherein the dielectric layer is configured to compress in response to an applied pressure, resulting in a change in a distance between the first inductive conductor and the second inductive conductor.

13. The pressure sensor according to claim 12, wherein an initial resonance frequency of the pressure sensor being determined in a non-compressed state of the dielectric layer, the initial resonance frequency being based on a dielectric constant of the dielectric layer and an initial separation distance between the first inductive conductor and the second inductive conductor.

14. The pressure sensor according to claim 13, wherein the applied pressure is determined by:
detecting a change in the initial resonance frequency caused by a capacitance change resulting from compression of the dielectric layer, and
using an external readout coil to compare the resonance frequency in the compressed dielectric layer state to the initial resonance frequency.

15. The pressure sensor according to claim 1, further comprising a resonant circuit electrically coupled to the first inductive conductor and the second inductive conductor, the resonant circuit being configured to exhibit a resonant frequency that varies as a function of an applied pressure to the dielectric layer.

16. The pressure sensor according to claim 15, wherein the resonant frequency is greater than a self-resonant frequency of a reader circuit to enable coupling for remote pressure detection.

17. The pressure sensor according to claim 1, wherein the first inductive conductor and the second inductive conductor are in direct contact with the dielectric layer.

18. The pressure sensor according to claim 1, wherein the dielectric layer comprises a plurality of pyramid-shaped structures.

19. A method for monitoring pressure changes within a living subject, the method comprising:
applying an external energy field to the pressure sensor of claim 1, the pressure sensor implanted in the living subject;
detecting a change in a resonant frequency of a resonant circuit of the pressure sensor;
correlating the change in the resonant frequency to a pressure within the living subject; and
displaying the pressure to a user to diagnose a condition of the living subject.

20. The method according to claim 19, wherein the living subject is a human.
